(19) United States
(12) Patent Application Publication
(10) Pub. No.: US 2008/0145380 A1
(43) Pub. Date: Jun. 19, 2008

(54) Algae and cancer treatment

(76) Inventor: Jane Teas, Columbia, SC (US)

Correspondence Address: The Weintraub Group, P.L.C., Suite 240, 32000 Northwestern Highway, Farmington Hills, MI, 48334, US

(21) Appl. No.: 12/002,451

(22) Filed: Dec. 17, 2007

Related U.S. Application Data
(60) Provisional application No. 60/875,183, filed on Dec. 15, 2006.

(51) Int. Cl. A61K 36/05; A61P 35/04; A61K 36/03

(52) U.S. Cl. 424/195.17

(57) ABSTRACT

A composition for primary prevention, treatment to slow progression of cancer or as an adjuvant therapy in treating cancer is defined by a mixture of a blue-green algae and a brown algae._These algae may be ingested simultaneously or serially. The composition may be formulated as a component of nutritional supplement. The composition and/or nutritional supplement may be ingested as a tablet, gel cap, emulsion, liquid, syrup or the like.

### CROSS-REFERENCE TO RELATED APPLICATIONS

This application is a completion application of co-pending U.S. provisional application Ser. No. 60/875,183, filed Dec. 15, 2006, the entire disclosure of which is hereby incorporated by reference.

### FIELD OF INVENTION

The present invention relates to the novel use of dietary algae to reduce cancer cell metabolism.

### BACKGROUND OF INVENTION

The epidemiologic evidence for seaweed as a chemopreventive food is very compelling. Although daily seaweed consumption is not uniform, even in Japan, people in Okinawa consume more seaweed and more pork, but have the lowest cancer incidence, mortality, and longest life spans¹. Using data from the 1970s, predating widespread westernization of the Japanese diet, Japanese women had one third the rate of pre-menopausal breast cancer and one ninth the rate of postmenopausal breast cancer². When a Japanese woman developed breast cancer, she was more likely to survive at least five years longer than a woman diagnosed with breast cancer in the United States^{3, 4}, and this seems to continue even after Japanese women immigrate to the US^{5, 6}. In a more recent study, Asian women who were born in the US have 60% higher risk of developing breast cancer than Asian women born in Asia⁷. This difference is even more striking when recent immigrants from Asia are compared to those who have been in the US for 10 or more years: their increased risk of developing breast cancer is 80% higher than Asian women who have lived in the US for less than 10 years. This argues strongly for an effect of environment and probably diet. Many food habits change with migration, including increased meat and milk, decreased vegetables, decreased rice, and especially decreased dietary seaweed.

In addition, other epidemiological studies of diet and cancer have yielded important correlates of dietary risks for developing cancer, such as high sugar, low fruit and vegetable intake, and high fat intakes, but there has only been one clinical study of a specific dietary intervention for people with diagnosed cancer. The first study by Thompson⁸ suggests that supplementation with flaxseed can improve the tumor characteristics (higher apoptosis, lower tumor cell proliferation, and decreased HER2 expression) even during a short period of time between breast cancer diagnosis and breast cancer surgery. Dietary manipulations after cancer diagnosis are welcome additions for cancer patients who would like to be able to increase their likelihood of survival by as many avenues as possible. Dietary algae, a common food in countries like Japan and Korea, where breast cancer rates are significantly lower than in the US, could have an effect on breast cancer cells in breast cancer patients.

Spirulina is a common dietary supplement. As food, it is currently used by people living along the shores of Lake Chad and was been used by the Aztecs about 500 years ago⁹. In Chad, the average daily dose of spirulina is between 9-12 g/d dry weight 10. Its efficacy in cancer treatment is less well researched than that of seaweeds, but nine studies have shown spirulina to induce cancer cell apoptosis¹¹⁻¹⁴, inhibit tumor cell proliferation^{11, 14, 15}, inhibit carcinogen metabolism^{16, 17}, inhibits tumor metastases in animals¹⁸, immune stimulation¹⁹⁻²¹, including two studies in humans, one showing immune enhancement²², and one showing inhibition of oral precancerous lesions²³. Most recently, inhibition of cyclooxygenase-2, leading to decrease prostaglandin production and upregulated Bcl-2 expression¹⁴ in normal cells treated with chemotherapy, and downregulated Bcl-2 in tumor cells¹³ have been identified, as well as caspase 3 activation²⁴. Cell cycle arrest has also been identified, leading to increased apoptosis of cancer cells but protection of normal cells¹². These studies show spirulina may have pro-apoptotic activity against tumor cells and be protective of healthy cells and whole animals.

This background forms the basis for further examination of the biologic basis of algae's inhibitory effects on breast cancer. An initial study was conducted in rats and was designed to answer the question of whether dietary seaweed could inhibit DMBA-induced mammary tumors. In a study of Laminaria angustata (one of the most commonly consumed seaweeds in Japan), there was a doubling in the time to developing the first tumor, and significantly fewer tumors per tumor bearing rat²⁵. Three other investigators, using two different species of brown seaweed (Laminaria religiosa^{26, 27}, and Undaria pinnatifida²⁸, all replicated the Teas study using seaweed as a protective dietary component against DMBA-induced cancers and reported similar protective effects.

### SUMMARY OF INVENTION

In accordance with the purposes of this invention, as embodied and broadly described herein, this invention, in one aspect, relates to the use of algae to reduce cancer cell metabolism. Additional advantages of the invention will be set forth in part in the description which follows or may be learned by practice of the invention.

For a more complete understanding of the present invention reference is made to the following detailed description and accompanying drawings, in which:

### DESCRIPTION OF THE FIGURES

The patent or application file contains at least one drawing executed in color. Copies of this patent or patent application publication with color drawings will be provided by the Office upon request and payment of the necessary fee.

FIG. 1 shows:

### TLR2 40X stereo biopsy

### TLR2 63X surgery

### TLR2 40X surgery

FIG. 2 shows:

### TLR4 40X stereo biopsy

### TLR4 40X surgery

### TLR4 40X surgery

FIG. 3 shows:

### TLR4 63X stereo biopsy

### TLR4 63X surgery

### TLR4 63X surgery

FIG. 4 shows:

### MDA-231 TLR2 63X:

FIG. 5 shows:

### MDA-231 TLR4 63X:

FIG. 6 shows:

### HCT-15 TLR2 63X:

FIG. 7 shows:

### HCT-15 TLR4 63X:

FIG. 8 shows:

### PR Stereo with DAPI 200x

### PR Surgery DAPI 200X

FIG. 9 shows:

### ER Stereo with DAPI 200X

### ER Surgery with DAPI 200X

FIG. 10 shows the effects of Spirulina and Undaria on HCT-15 TLR-4 protein expression.

FIG. 11 shows the effects of Spirulina and Undaria on MDA-MB-231 TLR-4 protein expression.

FIG. 12 shows the Changes in Urinary Urokinase Receptors for a study performed on three women with newly diagnosed breast cancer.

FIG. 13 shows:

### Control DAPI

### Seaweed plus Spirulina DAPI stain

### Control stained for urokinase

### Seaweed plus Spirulina stained for urokinase

FIG. 14 shows peak intensity in a SELDI study over a number of weeks.

### DETAILED DESCRIPTION OF INVENTION

It is to be understood by one of ordinary skill in the art that the present discussion is a description of exemplary embodiments only, and is not intended as limiting the broader aspects of the present invention.

The following experiments are provided to illustrate the present invention and are not intended to limit the scope of the invention. This invention claims compositions containing cyanobacteria and seaweed. It also claims a method of treating or preventing cancer in a subject, comprising administering the composition to the subject.

### Methods

Subjects with newly diagnosed needle biopsy proven breast cancer are recruited for a study of dietary algae. The study is conducted during the three week period between needle biopsy diagnosis and surgery. Subjects are given a baseline PET/CT scan, given algae (seaweed and spirulina), and return for a follow-up PET/CT scan one week later. Subjects continue to take the algae until 48 hours before their surgery.

On average, seaweed intake in Japan is estimated between 4 and 7 g/d dry weight²⁹. In Chad, spirulina comprises 9-12 g/d dry weight. Although there are only a few studies that address the appropriate therapeutic dose of algae in humans, a study by Sekiya extrapolated from the dose of seaweed needed to induce apoptosis of breast cancer cells to the human body and estimated that 5 g/d of seaweed would be sufficient³⁰. In the study by Mathew, the dose of 1 g of spirulina/d was sufficient to cause the complete regression of precancerous oral cancer lesions.²³ In this study, subjects are given 5 capsules of Undaria and 5 capsules of Spirulina per day. Each capsule contains 500 mg of the algae for a total of 2.5 grams of Undaria and 2.5 grams of spirulina per day.

PET scans are an imaging technique that uses radioactive labeled sugar to plot areas within the body that have abnormally high glucose uptake, indicating active cancer. It is ideal for assessing primary tumors, metastases, monitoring therapeutic changes associated with treatment and detecting tumor growth, as it measures reductions in glucose uptake by tumor cells within hours of administration of a therapy. The use of PET scans to determine response to chemotherapy and extent of cancer dissemination within the body has high specificity (88%-93%) and sensitivity (84-87%)³¹, and has accurately identified response to chemotherapy (between 88% and 91%)³² an average of 7 days after commencement of treatment.

Biological response is measured by histologic changes. A correlation is made between PET/CT scan results with changes seen in subsequent histology measures determined from breast cancer surgical specimens and changes between pre algae and post algae, and for a dose response as measured by duration of algal intake. Changes are measured in cancer cell morphology, immunochemistry staining, including estrogen receptor status (positive and negative), progesterone status (positive and negative), Ki-67 labeling index as a measure of cancer cell proliferation, Epstein Bar Virus, CXCR4, CCR5, Toll like receptors 2 and 4, p53 expression, MAP kinase pathway modulation (ERK phosphorylation), and expression of HER2, a cytoplasmic transmembrane receptor protein. In addition, analysis of urine for urokinase receptor levels was done pre and post the algae intervention.

In a similar dietary study using a flaxseed intervention for newly diagnosed breast cancer patients conducted during the time between breast cancer biopsy and surgery, Thompson reported significant reduction in cancer cell proliferation (Ki-67 labeling index) (18.1% controls vs. 12.6% flaxseed; p<0.001), increased cancer cell death (apoptosis index) (0.89% controls vs. 1.15% flaxseed; p<0.007), and decreased HER2 expression (0.47 pre vs. 0.34 post; p<0.003). Slight but not statistically significant increases in estrogen receptor (0.78 pre vs. 0.81 post) and progesterone receptor status (0.11 pre vs. 0.14 post) were seen in the flaxseed treatment, compared to non-significant decreases in these scores for the placebo arm (ER receptor status (0.72 pre vs. 0.65 post) and PR score (0.19 pre vs. 0.17 post))⁸. PET/CT scan confirmation of changes in cancer cell activity was not included in the Thompson study.

To further delineate the activity of seaweed, a pilot study of the impact of healthy postmenopausal women who consume 5 g/d of Undaria has been conducted. Profiling of serum and urinary proteins using surface enhanced laser desorption/ionization time of flight (SLEDI-TOF) mass spectrometry has become increasingly specific and can now identify with high sensitivity and specificity cancer types, including breast cancer status based on the specific changes in proteomic biomarkers. In this study, Recent studies (reviewed by Laronga³³ have shown that using SELDI-TOF can differentiate between BrCa1 carriers and healthy controls. These studies include the following: 1) 13/15 women with BrCa1 compared to one of the 15 non-carriers; 2) 14/16 patients with breast cancer even 6-9 months following treatment for breast cancer, compared to healthy controls; and 3) sentinel lymph node positive ( 22/27) patients from sentinel lymph node negative ( 55/71) patients. Array technology used for the discovery, validation, identification, and characteristics of disease-associated proteins from biological samples, such as SELDI ProteinChip® technology, is the primary proteomic platform technology for the NCI Early Detection Research Network (EDRN) study of early detection biomarkers of cancers (e.g., reviews by Grizzle et al.^{34 35}. In addition, array technology such as SELDI ProteinChip® technology has been used to identify changes in protein expression associated with the addition of novel foods, like green tea, to the diet³⁶. A seaweed urine SELDI study is shown in FIG. 14.

### Compositions

Disclosed are compositions comprising cyanobacteria and one or more types of seaweed. Both algae, a category that includes cyanobacteria (such as Spirulina) and brown seaweed, are nontoxic and safe. Spirulina has a long history of use by humans, both as food as a dietary supplement. Toxicity studies have shown it to be safe and to meet or exceed all national foods standards³⁷. The Food and Drug Administration classify brown seaweeds as “Generally Regarded As Safe” (GRAS)³⁸. It is eaten daily by millions of people around the world.

1. Cyanobacteria

The term “cyanobacteria,” as used herein, refers to prokaryotic organisms formerly classified as the blue-green algae. Cyanobacteria are a large and diverse group of photosynthetic bacteria which comprise the largest subgroup of Gram-negative bacteria. Cyanobacteria were classified as algae for many years due to their ability to perform oxygen-evolving photosynthesis³⁹. The cyanobacterium Arthrospira (“Spirulina”) has long been valued as a food source; it is high in protein, and can be cultivated easily. In tropical countries, it is a very important part of the diet, and was eaten regularly by the Aztecs; it is also served in several Oriental dishes. In the US, the popularity of Spirulina is primarily as a “health food,” being sold in stores as a dried powder or in tablet form.

2. Algae

Algae represent a large, heterogeneous group of primitive photosynthetic organisms which occur throughout all types of aquatic habitats and moist terrestrial environments. The term “algae,” as used herein, refers to Phaeophyta (brown algae) and Arthrospira (blue green algae known as “spirulina”). “Kelp” and “seaweed” are used interchangeably throughout. The kelps generally include the many large brown seaweeds and are among the most familiar forms found on North American coasts. Some have fronds up to 200 ft (61 m) long, e.g., the Pacific coast Nereocystis and Macrocystis. Common American species of kelp include Laminaria (kelp), Alaria (American wakame), Postelsia (sea palm), Ecklonia (paddleweed) Sargassum (gulfiveed), Fucus (rockweed), Pelvetia (bladder wrack), Macrocystis (giant bull kelp), Analipus (far needle), Nereocystis (giant kelp), and Ascophyllum (knotted wrack). In addition to its use as food, seaweeds are also commercial sources of potash, fertilizer, and medicines made from its vitamin and mineral content. Kelps are especially abundant in Japan, and many are included in the diet, including kombu (Laminaria), chigaiso (Alaria), hibamata (Fucus), kajime (Ecklonia), matsumo (Analipus/Heterochordaria), arame (Eisenia), hondawara (Sargassum), wakame (Undaria).

The safety of brown seaweeds depends on their iodine content 29. The popular Undaria (“wakame”), Alaria (American “wakame”) and the less commonly eaten Sargassum, have safe levels of iodine (40-100 ug/g). Other common brown seaweeds contain high iodine levels which could cause iodine sensitive individuals to develop transient thyrotoxicosis. The maximum tolerated dose of iodine is 1,000 μg/day, and the background level of iodine intake is about 250 μg/day. Iodine can be lowered by simple processing and storage techniques. In this invention, 5 grams of Undaria provides an additional 150-250 μg/day. Spirulina contains no iodine.

Algae, unlike narrowly targeted drugs, have been shown to exert a variety of health effects, including antiviral, antibacterial, antioxidant, anti-inflammatory, immune enhancing, probiotic, and cholesterol-lowering effects. As whole foods, rather than isolated fractions, the full spectrum of possible biochemical pathways for modulating health in diverse ways, are available to reduce breast, colon or other types of cancer. In addition to direct effects on cancer cells in culture, dietary algal extracts have shown a broad spectrum of immune enhancement in vivo and in vitro. These include increased production of interleukin-12 and interferon-1β in the presence of viral infection²², stimulated natural killer cell stimulation²², and B cell stimulation⁴⁰.

### Extracts, Derivatives, Lysates, and Fractions

Disclosed herein are compositions comprising fractions of cyanobacteria and one or more types of brown seaweed. Also disclosed are extracts, lysates, or derivatives of algae (Arthrospira and one or more types of brown seaweed). The extracts, lysates, and derivatives can be active or inactive.

The principal overall objective disclosed herein is to provide anti-tumorogenesis compositions, peptides and derivatives thereof, and broad medical uses thereof, including prophylactic and/or therapeutic applications against cancer.

In the practice of the present invention, the preferred composition is a mixture of spirulina and dietary brown seaweed, the brown seaweed being selected from the group consisting of: Laminaria, Alaria, Postelsia, Ecklonia, Sargassum, Fucus, Pelvetia, Macrocystis, Analipus, Nereocystic, Ascophyllum, Undaria and mixtures thereof.

The spirulina and the brown seaweed are preferably deployed as a powder. They are powderized by methods well known in the art, such as by milling, crushing or the like. They may be ingested serially, taken together, compounded into a tablet, pill, gelcap or added to a diluent to form a liquid version thereof either as a mixture of the two components or can be ingested individually.

### Supplements

Also disclosed herein are nutritional (also referred to as dietary throughout the application) supplements. A nutritional supplement is any compound or composition that can be administered to or taken by a subject to provide, supply, or increase an effect, such as an antiviral property. In one aspect, disclosed herein are nutritional supplements comprising any of the compositions disclosed herein. For example, a nutritional supplement can comprise a cyanobacteria (Arthrospira, or “spirulina”) and one or more types of brown seaweed, or fractions, extracts, lysates, or derivatives thereof. The nutritional supplement can comprise any amount of the compositions disclosed herein, but will typically contain an amount determined to supply a subject with a desired dose of the composition. The exact amount of composition required in the nutritional supplement will vary from subject to subject, depending on the species, age, weight and general condition of the subject, the severity of the dietary deficiency being treated, the particular mode of administration, and the like. Thus, it is not possible to specify an exact amount for every nutritional supplement. However, an appropriate amount can be determined by one of ordinary skill in the art using only routine experimentation given the teachings herein.

In one specific example, a nutritional supplement can comprise from about 1 to about 20 grams of brown seaweed, and 1 to about 20 grams of Arthrospira (“spirulina”), or fractions, extracts, lysates, or derivatives thereof where any of the stated values can form an upper or lower endpoint when appropriate. Furthermore, the brown seaweed and cyanobacteria (Arthrospira or “spirulina”) or fractions, extracts, lysates, or derivatives thereof can be given in the same supplement, or simultaneously in different supplements, or in adjacent supplements taken near the same time, such as within about 10, 20, 30, 40, or 50 seconds, or within about 1, 2, 3, 4, 5, 6, 7, 8, 9, 10, 11, 12, 13, 14, 15, or 30 minutes, or within about 1, 2, 3, 4, 5, 6, 7, 8, 9, 10, 11 or 12 hours, or within 24 hours. Also, different types of algae can be administered simultaneously. Any types of algae known to those of skill in the art can be administered according to the methods disclosed herein.

The nutritional supplement can also comprise other nutrient(s) such as vitamins other trace elements, minerals, and the like. Further, the nutritional supplement can comprise other components such as preservatives, antimicrobials, anti-oxidants, chelating agents, thickeners, flavorings, diluents, emulsifiers, dispersing aids, or binders.

The nutritional supplements are generally taken orally and can be in any form suitable for oral administration. For example, a nutritional supplement can typically be in a tablet, gel-cap, capsule, liquid, sachets, or syrup form. Taking such nutritional supplements is beneficial to treat or prevent cancer.

### Results

The effects of the combination of Undaria and spirulina on two receptor sites for the innate immune system, TLR-2 and TLR-4 are examined. Additional studies of the associated expression of urokinase receptor are also presented. This study demonstrates that these receptors are on both MDA-MB-231 breast cancer cells and on human breast cancer tissue removed during biopsy and surgery, and changes in urinary excretion of urokinase receptor can be seen if the patient had initial high levels of urokinase receptors. Differences in cell architecture is demonstrated through a comparison of the “sterotactic biopsy” with the “surgical specimen” in the tissue samples from a newly diagnosed breast cancer patient who took seaweed plus spirulina for a week between biopsy and surgery.

To illustrate, and as shown in FIGS. 4-7, colon and breast cancer cell cultures, from the American Type Culture Collection, were stained for two of the receptors, TLR-2 and TLR-4, important in the innate immune system.

Cell line: The MDA-MB-231 cell line was purchased from the American Type Culture Collection (Manassas, Va.) and was maintained in RPMI-1640 media (Sigma-Aldrich), containing 10% FBS with 100 u/ml penicillin and 100 μg/ml streptomycin in a humidified atmosphere with 5% CO2.

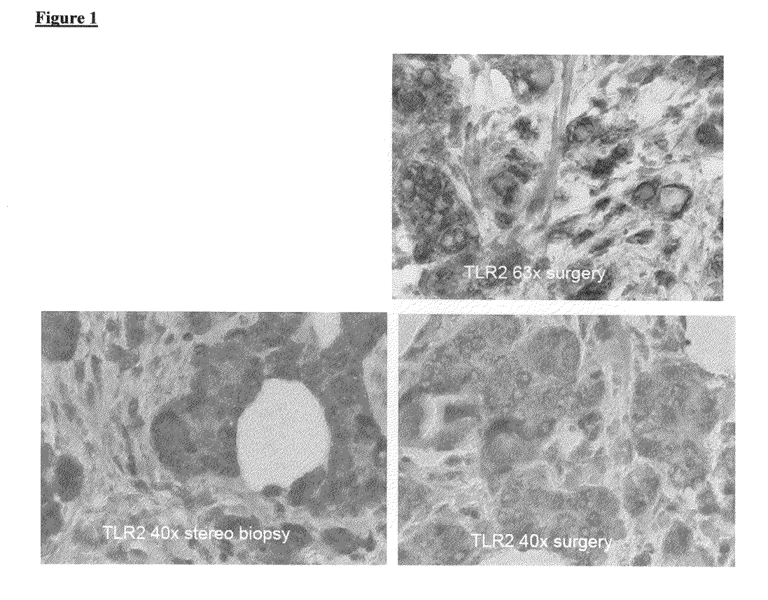

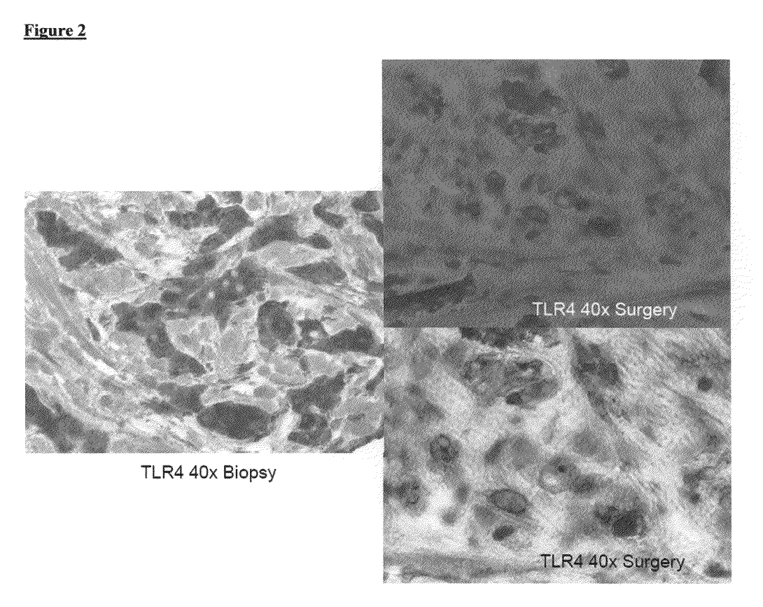

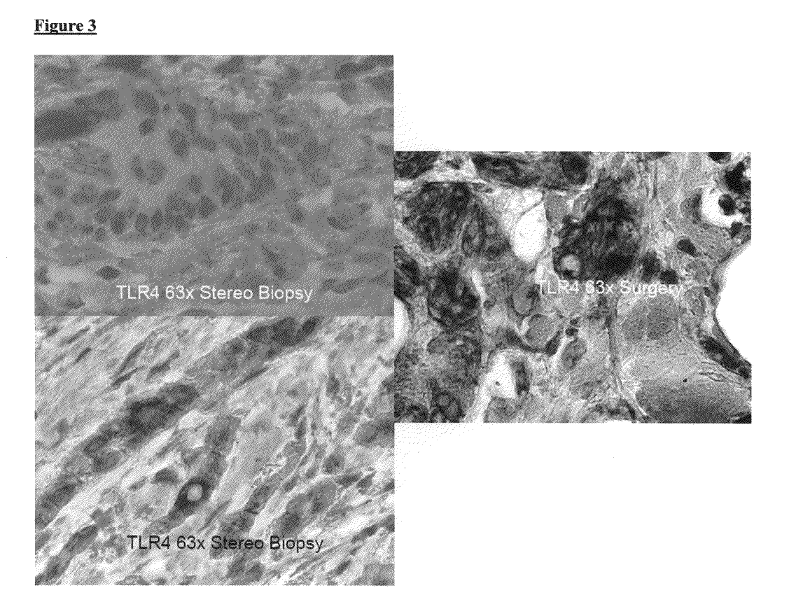

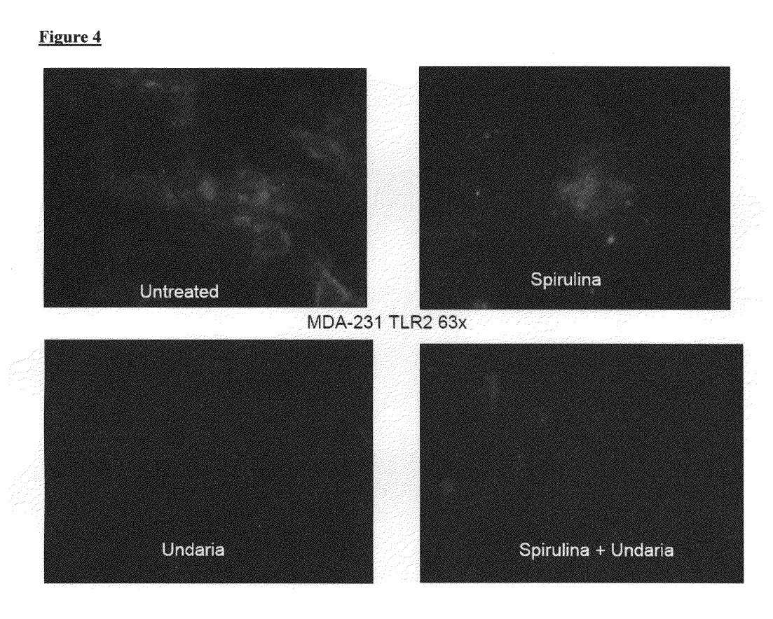

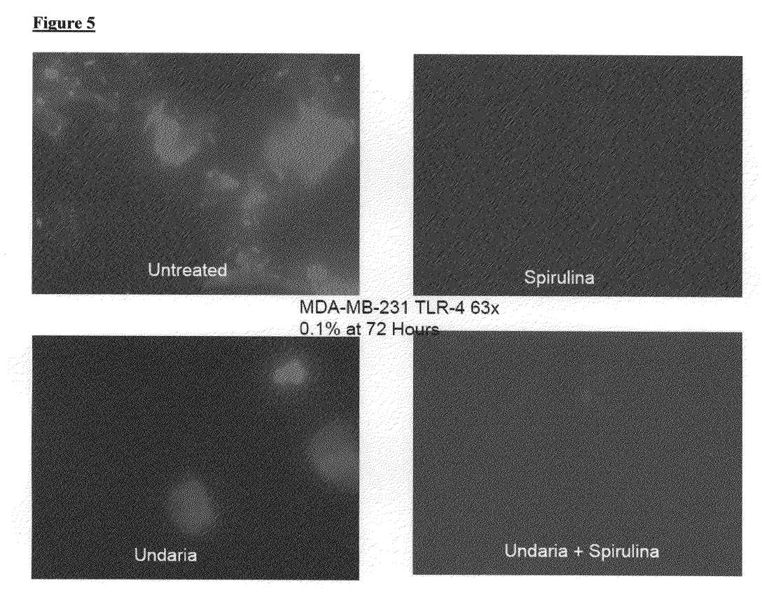

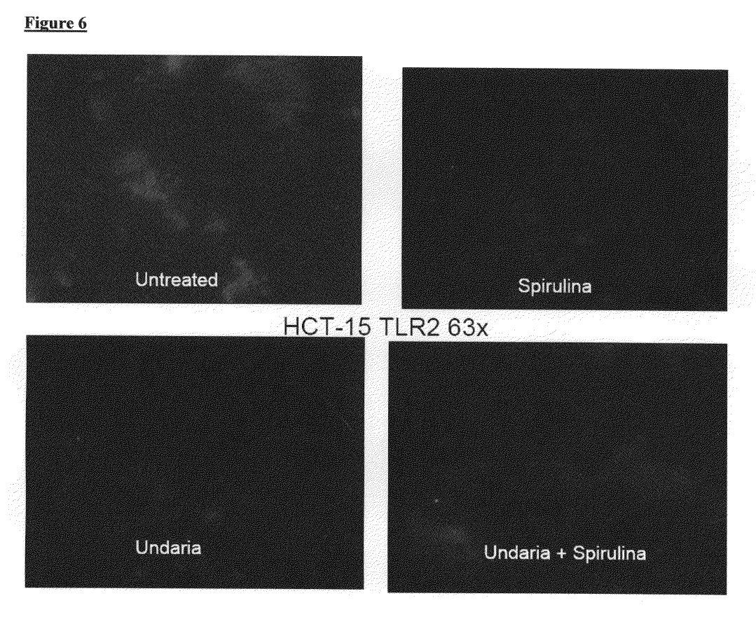

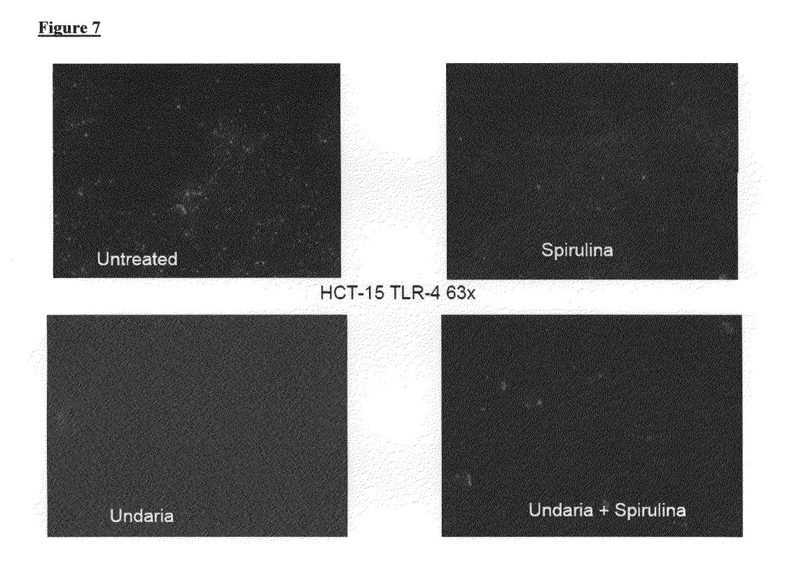

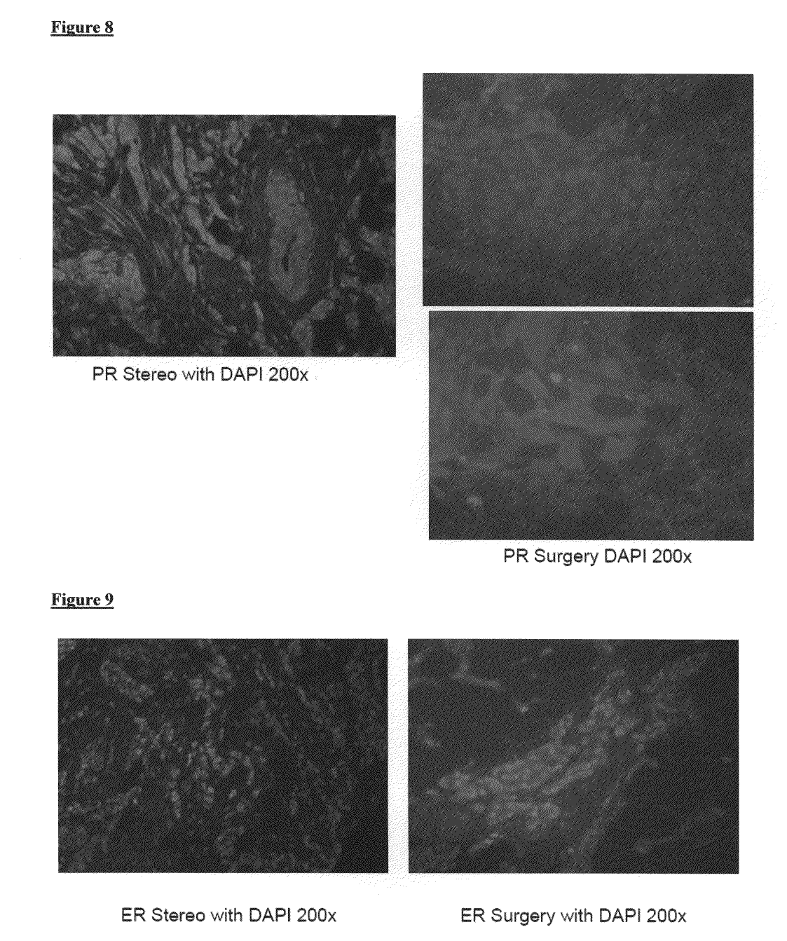

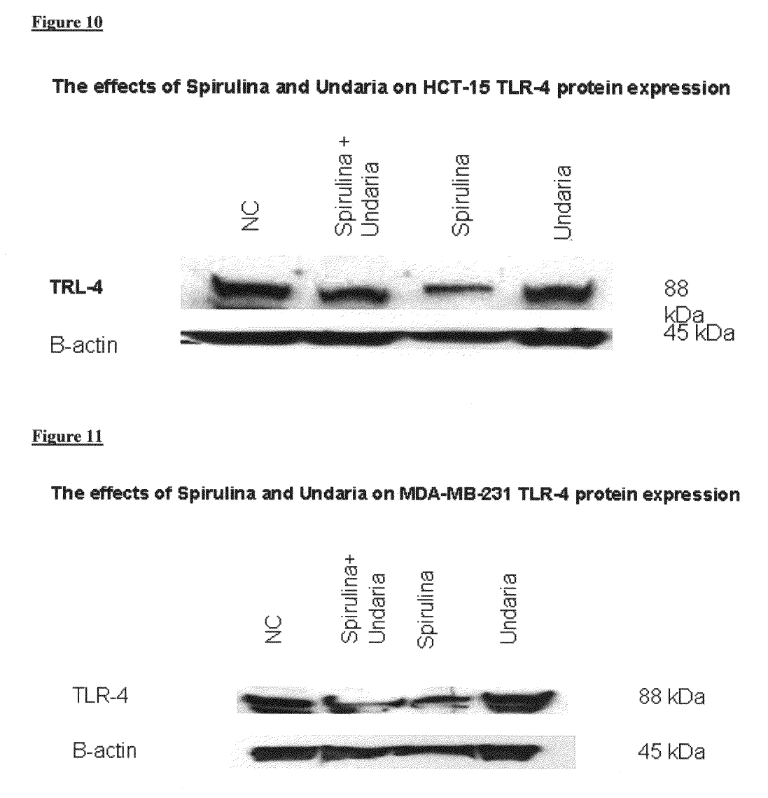

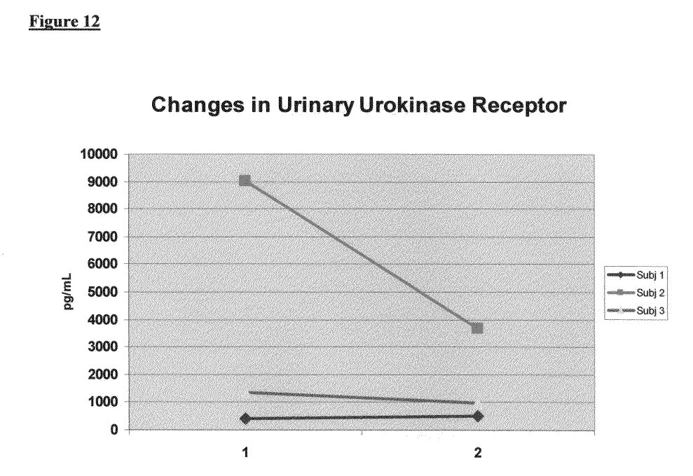

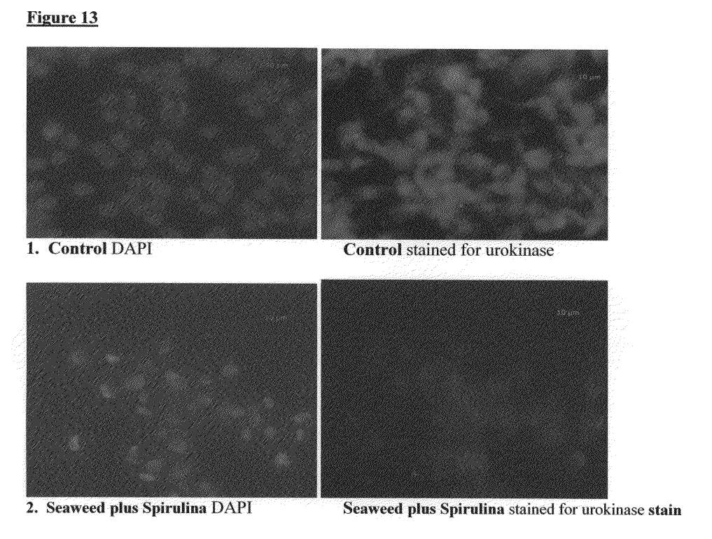

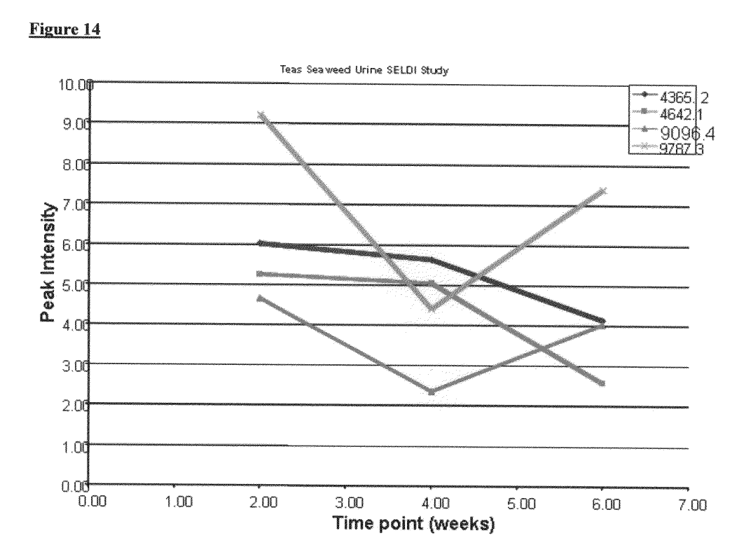

As far as it is known in the art, this is the first time any effect of algae has been shown on breast cancer cells. The comparison of these cell cultures confirms that seaweed (Undaria) blocks or down regulates or causes to internalize the TLR-2 receptors, while spirulina blocks or down regulates or causes to internalize TLR-4 receptors. The combination of seaweed and spirulina blocks both receptors making them unavailable for bacteria and viruses and having a profound effect on inflammatory responses. When these receptors are not blocked, the cancer cells subvert the normal inflammatory receptors (TLR-2 and TLR-4) (FIGS. 1-3, 10,11), and use these receptors to create inflammation in the tumor microenvironment. This innate immune inflammatory response is sufficient to promote immunotolerance for tumor cells, allowing them to progress. The inflammation surrounding the cancer cells sends a signal to the lymphocytes to tolerate the inflammation, rather than to attack the cells. This absence of lymphocytic response increases the tumorogenesis in the tissue. In addition, a downstream receptor to the TLR activation pathway (the urokinase receptor) is also directly affected by algae. In the three women who took the algae supplement, one had high urokinase receptor levels. High urokinase receptor levels are associated with faster progression of breast cancer. In published studies, urokinase levels do not change from time of diagnosis to death. However, for the woman in the study who had high urokinase levels, there was more than a third decrease with seaweed supplementation. No effects were noted for the women with normal levels of urinary urokinase. Therefore, altered cell-surface receptor responses through the administration of algae have important implications for treating and/or preventing breast cancer. This is graphically shown in FIG. 12.

The four slides in FIG. 13 show 4′-6-Diamidino-2-phenylindole (DAPI) forms fluorescent complexes with natural double-stranded DNA. It is used here to indicate the presence of cancer cells. The cells were treated with either control (normal cell media) or algae. The same microscopic field of cancer cells is seen in each horizontal line. The lack of fluorescent complexes when treated with algae (seaweed plus spirulina) (#2) but not when treated with control media (#1) supports the idea that algae competitive binds to the urokinase receptor.

In addition, results from the histological studies from the breast cancer subjects show the algae mix increased estrogen receptor positivity in the cancer cells (FIGS. 8,9). Estrogen receptor positive cancer is treatable with reasonable success by chemotherapy. Estrogen receptor negative cancer is not as responsive to chemotherapy or hormonal therapy. Giving the seaweed/spirulina combination to patients may make drugs like Tamoxifen more effective. Tamoxifen is an estrogen inhibitor. The worst prognosis is for women with estrogen independent (ER-) breast cancer, because estrogen inhibitors do not work. By way of illustration, three subjects with newly diagnosed breast cancer underwent a PET/CT scan and were administered seaweed+spirulina for a week, and then underwent a repeat PET/CT scan. Comparing the tissue samples from the breast tumor biopsy and the breast tumor excised during surgery shows a change in ER and PR positivity. (FIGS. 8,9) The results from CXCR4, Her2neu TLR2 and TLR4 (FIGS. 8-11), and Ki67 (indicative of cell proliferation) are shown as well.

### Significance

Breast cancer is a leading cause of death for women. Administration of a seaweed/spirulina combination to patients makes drugs like Tamoxifen more effective. Tamoxifen is an estrogen inhibitor, and the worst prognosis is for women with estrogen independent (ER-) breast cancer, because estrogen inhibitors do not work. Further, the seaweed/spirulina combination blocks receptors of the innate immune system and alters CXCR4 and CCR5 receptors. By preventing the innate immune inflammatory response, the likelihood of tumor formation is decreased and escape from immunesurveillance is reduced. CXCR4 and CCR5 receptors are key to metastatic cancer cell, and the combination of spirulina and seaweed is likely to inhibit cancer metastases by decreasing the number of receptors available on the cancer cell surface for adhesion. The clinical importance of decreasing urokinase in women who have high urokinase levels is highly significant in preventing breast cancer progression.

### REFERENCES

1. Teas J. The dietary intake of Laminaria, a brown seaweed, and breast cancer prevention. Nutr Cancer 1983; 4: 217-22.
2. Reddy B S, Cohen L A, McCoy G D, Hill P, Weisburger J H, Wynder E L. Nutrition and its relationship to cancer. Adv Cancer Res 1980; 32: 237-345.
3. Wynder E L, Kajitani T, Kuno J, Lucas J C J, Depalo A, Farrow J. A comparison of survival rates between American and Japanese patients with breast cancer. Surg Gynecol Obstet 1963; 117:196-200.
4. Morrison A S, Black M M, Lowe C R, MacMahon B, Yuasa S. Some international differences in histology and survival in breast cancer. International Journal of Cancer 1973; 11:261-7.
5. Kanemori M, Prygrocki M. Results of breast conservation therapy from a single-institution community hospital in Hawaii with a predominantly Japanese population. Int J Radiat Oncol Biol Phys 2005; 62(1):193-7.
6. Pineda M D, White E, Kristal A R, Taylor V. Asian breast cancer survival in the US: a comparison between Asian immigrants, US-born Asian Americans and Caucasians. Int J Epidemiol 2001; 30(5):976-82.
7. Ziegler R G, Hoover R N, Pike M C, et al. Migration patterns and breast cancer risk in Asian-American women. J Natl Cancer Inst 1993; 85:1819-27.
8. Thompson L U, Chen J M, Li T, Strasser-Weippi K, Goss P E. Dietary flaxseed alters tumor biological markers in postmenopausal breast cancer. Clin Cancer Res 2005; 11(10):3828-35.
9. Belay A. The potential application of Spirulina (Arthrospira) as a nutritional and therapeutic supplement in health management. J Am Nutraceut Assoc 2002; 5(2):27-48.
10. Abdulqader G, Barsanti L, Tredici M R. Harvest of Arthrospira platensis from Lake Kossorom (Chad) and its household usage among the Kanembu. JOURNAL OF APPLIED PHYCOLOGY 2000; 12(3-5):493-8.
11. Wu L C, Ho J A, Shieh M C, Lu I W. Antioxidant and antiproliferative activities of Spirulina and Chlorella water extracts. J Agric Food Chem 2005; 53(10):4207-12.
12. Subhashini J, Mahipal S V, Reddy M C, Mallikarjuna Reddy M, Rachamallu A, Reddanna P. Molecular mechanisms in C-Phycocyanin induced apoptosis in human chronic myeloid leukemia cell line-K562. Biochem Pharmacol 2004; 68(3):453-62.
13. Reddy M C, Subhashini J, Mahipal S V, et al. C-Phycocyanin, a selective cyclooxygenase-2 inhibitor, induces apoptosis in lipopolysaccharide-stimulated RAW 264.7 macrophages. Biochem Biophys Res Commun 2003; 304(2):385-92.
14. Liu X M, Zhang HQ. [Effect of polysaccharide from Spirulina platensis on hematopoietic cells proliferation, apoptosis and Bcl-2 expression in mice bearing tumor treated with chemotherapy]. Chinese. Yao Xue Xue Bao 2002; 37(8):616-20.
15. Chen F, Zhang Q. [Inhibitive effects of spirulina on aberrant crypts in colon induced by dimethylhydrazine] Chinese. Zhonghua Yu Fang Yi Xue Za Zhi 1995; 29(1):13-7.
16. Dasgupta T, Banejee S, Yadav P K, Rao A R. Chemomodulation of carcinogen metabolising enzymes, antioxidant profiles and skin and forestomach papillomagenesis by Spirulina platensis. Mol Cell Biochem 2001; 226 (1-2):27-38.
17. Mittal A, Kumar P V, Banerjee S, Rao A R, Kumar A. Modulatory potential of Spirulina fusiformis on carcinogen metabolizing enzymes in Swiss albino mice. Phytother Res 1999; 13(2):111-4.
18. Mishima T, Murata J, Toyoshima M, et al. Inhibition of tumor invasion and metastasis by calcium spirulan (Ca-SP), a novel sulfated polysaccharide derived from a blue-green alga, Spirulina platensis. Clin Exp Metastasis 1998; 16(6):541-50.
19. Shklar G, Schwartz J. Tumor necrosis factor in experimental cancer regression with alphatocopherol, beta-carotene, canthaxanthin and algae extract. Eur J Cancer Clin Oncol 1988; 24(5):839-50.
20. Pugh N, Ross S A, ElSohly H N, ElSohly M A, Pasco D S. Isolation of three high molecular weight polysaccharide preparations with potent immunostimulatory activity from Spirulina platensis, aphanizomenon flos-aquae and Chlorella pyrenoidosa. Planta Med 2001; 67(8):737-42.
21. Guan Y, Guo B. [Inhibition activity of spirulina platensis proteins photo-immobilization biomaterial on proliferation of cancer cells] Chinese. Sheng Wu Yi Xue Gong Cheng Xue Za Zhi 2002; 19(1):1-3.
22. Hirahashi T, Matsumoto M, Hazeki K, Saeki Y, Ui M, Seya T. Activation of the human innate immune system by Spirulina: augmentation of interferon production and NK cytotoxicity by oral administration of hot water extract of Spirulina platensis. Int Immunopharmacol 2002; 2(4):423-34.
23. Mathew B, Sankaranarayanan R, Nair P P, et al. Evaluation of chemoprevention of oral cancer with Spirulina fusiformis. Nutr Cancer 1995; 24(2):197-202.
24. Pardhasaradhi B V, Ali A M, Kumari A L, Reddanna P, Khar A. Phycocyanin-mediated apoptosis in AK-5 tumor cells involves down-regulation of Bcl-2 and generation of ROS. Mol Cancer Ther 2003; 2(11):1165-70.
25. Teas J, Harbison M L, Gelman R S. Dietary seaweed (Laminaria) and mammary carcinogenesis in rats. Cancer Res 1984; 44(7):2758-61.
26. Yamamoto I, Maruyama H, Moriguchi M. The effect of dietary seaweeds on 7,12-dimethyl-benz[a]anthracene-induced mammary tumorigenesis in rats. Cancer Lett 1987; 35(2):109-18.
27. Maruyama H, Watanabe K, Yamamoto I. Effect of dietary kelp on lipid peroxidation and glutathione peroxidase activity in livers of rats given breast carcinogen DMBA. Nutr Cancer 1991; 15(3-4):221-8.
28. Funahashi H, Imai T, Tanaka Y, et al. Wakame seaweed suppresses the proliferation of 7,12-dimethylbenz(a)-anthracene-induced mammary tumors in rats. Jpn J Cancer Res 1999; 90(9):922-7.
29. Teas J, Pino S, Critchley A, Braverman L E. Variability of Iodine Content in Common Commercially Available Edible Seaweeds. Thyroid 2004; 14(10):836-41.
30. Sekiya M, Funahashi H, Tsukamura K, et al. Intracellular signaling in the induction of apoptosis cancer cell line by water extract of Mekabu. Int J Clin Oncol 2005; 10:122-6.
31. Gambhir S S, Czernin J, Schwimmer J, Silverman D H S, Coleman R E, Phelps M E. A tabulated summary of the FDG PET literature. J Nucl Med 2001; 42:1S-93S.
32. Schelling M, Avril N, Nährig J, et al. Positron Emission Tomography Using [18F]Fluorodeoxyglucose for Monitoring Primary Chemotherapy in Breast Cancer. Journal of Clinical Oncology 2000; 18(8): 1689-95.
33. Laronga C, Becker S, Watson P, et al. SELDI-TOF serum profiling for prognostic and diagnostic classification of breast cancers. Dis Markers 2003; 19(4-5):229-38.
34. Grizzle W E, Semmes O J, Basler J, et al. The early detection research network surface-enhanced laser desorption and ionization prostate cancer detection study: A study in biomarker validation in genitourinary oncology. Urol Oncol 2004; 22(4):337-43.
35. Zhang Z, Bast R C J, Yu Y, et al. Three biomarkers identified from serum proteomic analysis for the detection of early stage ovarian cancer. Cancer Res 2004; 64(16):5882-90.
36. Tsuneki H, Ishizuka M, Terasawa M, Wu J-B, Sasaoka T, Kimura I. Effect of green tea on blood glucose levels and serum proteomic patterns in diabetic (db/db) mice and on glucose metabolism in health humans. BMC Pharmacology 2004; 4(18):1-18.
37. Food and Drug Administration F. GRAS Notice No. GRN 000127. In: Office of Food Additive Safety CfFSaAN, ed.; 2003.
38. Food and Drug Administration F. GRAS status of certain red and brown algae and their extractives. Federal Register 1982; 47(207):47373-6.
39. Vonshak A, ed. Spirulina platensis (Arthrospira) Physiology, cell-biology and biotechnology. Bristol: Taylor & Francis Lts.; 1997.
40. Shan B E, Yoshida Y, Kuroda E, Yamashita U. Immunomodulating activity of seaweed extract on human lymphocytes in vitro. Int J Immunopharmacol 1999; 21(1):59-70.

Having, thus, described the invention, what is claimed is:

1. A composition for use in the treatment of cancer comprising:
(a) an effective amount of a blue-green algae, and
(b) an effective amount of a brown algae having safe levels of iodine.

2. The composition of claim 1 wherein:
the blue-green algae is a cyanobacteria.

3. The composition of claim 1 wherein the brown algae is selected from the group consisting of Agarum, Sargassum, Undaria, Alaria and mixtures thereof.

4. The composition of claim 1 wherein:
(a) the blue-green algae is a cyanobacteria, and
(b) the brown algae is selected from the group consisting of Agarum, Sargassum, Laminaria, Undaria, Alaria and mixtures thereof.

5. The composition of claim 4 wherein:
(a) the blue-green algae is present in an amount ranging from about one to about three thousand parts thereof based on the total weight of the composition, and
(b) the brown algae is present in an amount ranging from about one to about three thousand parts thereof based on the total weight of the composition.

6. The composition of claim 5 wherein the composition comprises a nutritional supplement, the supplement including at least one of a vitamin, a mineral, a preservative, an antimicrobial agent, an anti-oxidant, a chelant, a thickener, a flavoring and mixtures thereof.

7. The composition of claim 6 which further includes a diluent, an emulsifier, a dispersant, a binder and mixtures thereof.

8. The composition of claim 7 wherein the composition is either a tablet, a gel-cap, a capsule, a liquid, a sachet or a syrup.

9. The composition of claim 4 wherein:
(a) the blue-green algae is present as either the cyanobacteria, a fraction, an extract, a lysate or a derivative thereof, and
(b) the brown algae is present as either the brown algae, a fraction, an extract, a lysate or a derivative thereof.

10. A nutritional supplement, comprising the composition of claim 1.

11. The nutritional supplement of claim 10 which further includes at least one adjuvant selected from the group consisting of a vitamin, a mineral, a preservative, an antimicrobial agent, an anti-oxidant, a chelant, a thickener, a flavoring and mixtures thereof.

12. The nutritional supplement of claim 11 wherein:
(a) the blue-green algae is present in an amount ranging from about one to about three thousand parts thereof based on the total weight of the composition, and
(b) the brown algae is present in an amount ranging from about one to about three thousand parts thereof based on the total weight of the composition.

13. The nutritional supplement of claim 12 wherein the supplement is present as either a tablet, gel cap, emulsion, syrup,

14. A method for treating cancer, comprising:
orally ingesting the composition of claim 1.

15. The method of claim 14 which further comprises formulating the composition as a nutritional supplement.

16. The method of claim 15 wherein the components of the composition are ingested serially in a time period of from about one to about 24 hours between ingestion of the first component and the second component.

17. The composition of claim 1 wherein:
the blue-green algae is Arthrospira.
